(19) United States
(12) Patent Application Publication
(10) Pub. No.: US 2013/0103020 A1
(43) Pub. Date: Apr. 25, 2013

(54) Cryosurgical system

(71) Applicant: Alexander Levin, Binyamina (IL)

(72) Inventor: Alexander Levin, Binyamina (IL)

(21) Appl. No.: 13/317,502

(22) Filed: Oct. 20, 2011

(51) Int. Cl. A61B 18/02

(52) U.S. Cl. 606/24; 606/23; 606/26

(57) ABSTRACT

An invention relates to the area of cryosurgical equipment. It proposes a cryosurgical system, which incorporates measuring and computing means for estimation of a real time ice ball diameter and operation temperature of a cryotip (the distal section of a cryosurgical probe). The cryosurgical probe of the cryosurgical system operates by blowing in a gaseous medium at cryogenic temperature.

### STATEMENT REGARDING FEDERALLY SPONSORED RESEARCH

Not applicable.

### FIELD OF THE INVENTION

This invention relates to the field of cryosurgical equipment and, particularly, to cryosurgical systems intended to perform internal cryogenic treatments.

### BACKGROUND OF THE INVENTION

Common cryosurgical instruments can be divided into two categories:

a) Cryosurgical devices operating with application of a liquid refrigerant (or refrigerant in the form of liquid-gaseous mixture) such as liquid nitrogen or liquid argon;
b) Cryosurgical devices operating on the base of expansion of highly pressurized gas (Joule-Thomson effect).

Each of these categories of cryosurgical equipment has its advantages and drawbacks. However, none of this cryosurgical equipment allows to estimate the right now temperature of a cryotip (the distal section of a cryosurgical probe) and the real time ice ball diameter formed around the cryotip. Application of a thermocouple, which is in a good thermal contact with the internal surface of the cryotip, for measuring the temperature of the cryotip presents a complicated technical problem. Ultrasound imaging devices, which are commonly used to estimate the diameter of an ice ball formed around the cryotip, require adequate skill and ability of a surgeon executing a cryosurgical procedure.

Application of gaseous medium at cryogenic temperatures as means for performance of cryosurgical procedures is mentioned in U.S. Pat. No. 5,254,116. The authors of this patent hold that “for miniature cryoprobes cold nitrogen gas or helium gas is often used instead of liquid nitrogen. This compromise has proven unsatisfactory because the heat transfer efficiency between the gas and the probe tip in contact with the tissue is very low and consequently, the ice ball created at the tip is not large enough and the temperature is not low enough for typical clinical applications. Moreover, the pressure used for such probes is often in excess of 500 or 600 psi, which requires special safeguards to prevent potential hazards”.

However, calculated values of heat transfer coefficient for gaseous helium at cryogenic temperatures, demonstrate that for sufficiently small hydraulic diameter of the cryotip, it is possible to achieve heat transfer coefficient of 1000 W/m²° C. or more (see: S. G. Kandlikar et al. HEAT TRANSFER AND FLUID FLOW IN MINICHANNELS AND MICROCHANNELS, ELSEVIER 2006, Chapter I and Chapter III).

Additional increase of heat transfer coefficient for gaseous medium can be achieved by enhancing the internal surface of the cryotip.

On the other hand, application of the gaseous medium at cryogenic temperature as means for performance of cryosurgical procedures, provides some advantages in controlling and measuring of an ice ball formation; these advantages cannot be obtained with the other cryosurgical systems mentioned above.

### BRIEF SUMMARY OF THE INVENTION

A proposed cryosurgical system with a built-in control unit for estimation of some parameters related to ice-ball formation around a cryotip consists of following means for calculation of the cryotip temperature and the ice-ball diameter: a mass flow rate measuring sub-unit, means for measuring temperature of the gaseous medium before its introduction into cryosurgical probe and temperature of the gaseous medium immediately after its escape from the cryosurgical probe. It gives possibility to calculate amount of heat transferred from the surrounding tissue into cryotip and according to this value to estimate the diameter of an ice ball formed around the cryotip. Moreover, mass flow rate value is presented indirectly in dimensionless Reynolds number.

Therefore, heat transfer coefficient of the internal surface of the cryotip for the flow of a specific gaseous medium can be estimated as a function of the mass flow rate.

Heat rate, which is transferred through the internal wall of the cryotip, can be calculated in two ways:

Q=M·(Tₒᵤₜ−Tᵢₙ−ΔT_{shaft})·C (1)

where Q—heat rate, M—mass flow rate, C—specific heat of gaseous medium, Tₒᵤₜ—temperature of escaped gaseous medium, Tᵢₙ—temperature of supplied gaseous medium, ΔT_{shaft}—systematic temperature error caused by heat transfer through the cryoprobe shaft.

Q=K·S·[T_{w}−(Tₒᵤₜ+Tᵢₙ−ΔT_{shaft})/2] (2),

where: K—heat transfer coefficient, S—the internal surface area of the cryotip. Instead of T_{w}−(Tₒᵤₜ+Tᵢₙ−ΔT_{shaft})/2 the log mean temperature difference can be calculated.

Heat transfer coefficient K is a function of (Tₒᵤₜ+Tᵢₙ−ΔT_{shaft})/2, which can be found by testing or theoretically calculated, and its values are recorded in the control unit memory. The change of heat transfer coefficient K with changing average temperature of the gaseous medium is caused by variation of viscosity and thermal conductivity of the gaseous medium with the temperature varying.

It should be noted that ΔT_{shaft} is a function of (Tₒᵤₜ+Tᵢₙ)/2; however, as a first approximation for vacuum thermal insulation of the cryoprobe shaft, this systematic temperature error can be assumed to be constant and measured experimentally by a preliminary testing procedure.

It allows calculating the temperature of the internal surface of the cryotip as:

T_{w}=[M·(Tₒᵤₜ−Tᵢₙ−ΔT_{shaft})·C+K·S·(Tₒᵤₜ+Tᵢₙ−ΔT_{shaft})/2]/K·S (3)

This calculated value of the cryotip temperature allows to estimate final diameter of the formed ice ball; the estimation may be done analytically or by empirical data in form of diagrams or nomograms and recorded in the control unit memory (such data is presented, for example, in the book V. G. Vedenkov et al. CRYOGENIC MEDICAL TECHNIQUE, METHODICAL RECOMMENDATIONS, Moscow 1991, in Russian).

Moreover, changing T_{w} over a cryosurgical operation period, allows to indicate stagnation in changing T_{w} and to cease the cryosurgical operation or to do alternation to a thawing mode (if the cryosurgical operation should be performed by two or more cycles freezing-thawing).

In addition, this method of a cryotip temperature measuring can be applied for thawing control, when the gaseous medium at temperature above 0° C. serves for thawing an ice ball formed around the cryotip. In this case, calculated temperature of the cryotip indicates the absence or presence of adhesion between the cryotip and the ice ball.

In such a way, the proposed cryosurgical system (without the cryoprobe itself) consists of some basic units: a source of pressurized gaseous medium, preferably, helium at cryogenic temperature; a flexible thermo-insulated hose (preferably with vacuum thermo-insulation); at least two temperature sensors, which are measuring Tₒᵤₜ—temperature of escaped gaseous medium and Tᵢₙ—temperature of supplied gaseous medium; a mass flowmeter for measuring mass flow rate of the gaseous medium (preferably helium); a control unit with recorded data regarding estimated final diameters of the formed ice balls as a function of final temperature of the cryotip and thermo-physical properties of the tissue to be destroyed by cryosurgical operation.

There are five main designs of the source of pressurized gaseous medium at cryogenic temperature:

1. Combination of a bottle with pressurized gaseous medium and a Dewar flask with an embedded heat exchanger; the pressurized gaseous medium is supplied from the bottle into the embedded heat exchanger via a supply line, which is provided with an installed mass flow rate gauge. The pressurized gaseous medium at cryogenic temperature exits the heat exchanger and enters the proximal section of a central feeding conduit, which is situated in the internal space of the Dewar flask. The distal external end of the central feeding conduit should be coupled with a flexible thermo-insulated hose.

2. Combination of a bottle with pressurized gaseous medium and a Dewar flask with an external heat exchanger of recuperative type; the pressurized gaseous medium is supplied from the bottle into the external heat exchanger via a supply line, which is provided with an installed mass flow rate gauge.

At the same time, liquid-gaseous mixture of cryogen is supplied into the external heat exchanger. The pressurized gaseous medium at cryogenic temperature exits the heat exchanger and enters the proximal section of a central feeding line of the flexible thermo-insulated hose. The external heat exchanger is provided with an external thermo-insulation (preferably—with a vacuum thermo-insulation). In another preferable embodiment the flexible hose and the external heat exchanger have a common vacuum thermo-insulation. Evaporated cryogen is cleared out of the heat exchanger into the atmosphere or into an additional heat exchanger of recuperative type for precooling the pressurized gaseous medium.

3. Evaporation of liquid cryogen in the Dewar flask under a certain pressure provides the pressurized gaseous medium at cryogenic temperature; the pressurized gaseous medium is entering into the open proximal end of the central feeding conduit.

4. There are two bottles with pressurized gases; the first one serves as a source of the pressurized gaseous medium, which cools the cryotip to cryogenic temperature; and the second one serves as a source of highly pressurized gas being expanded via an orifice tube in a heat exchanger; the expansion causes the cooling of the gas on account of Joule-Thomson effect with following cooling of the pressurized gaseous medium in the counter-flow heat exchanging unit arranged in the heat exchanger.

5. There is a combination of two cooling sources for lowering the temperature of the pressurized gaseous medium to a cryogenic temperature. The first source is a liquid cryogen, which is contained in a Dewar flask and serves for preliminary cooling of a highly pressurized gas below its inversion temperature (when the Joule-Thomson (Kelvin) coefficient μ_{JT} is negative). This cooled highly pressurized gas serves for further lowering its temperature by its expansion via an orifice tube with Joule-Thomson effect and following cooling of the pressurized gaseous medium in the heat exchanger as it is described in the version four.

In the second version mentioned above, the lower internal section of the Dewar flask is provided with an electrical heater; a wattmeter is measuring the rate of heating the liquid cryogen. It allows to transform a value measured by the wattmeter into the mass flow rate of the pressurized gaseous medium.

In a preferable design of the flexible thermo-insulated hose with a vacuum thermo-insulation, the distal section of the central feeding conduit is provided with a vacuum thermo-insulation, which is common with the vacuum thermo-insulation of the central conduit of the flexible thermo-insulated hose. It allows minimizing heat transfer from the surroundings to this central conduit.

The central feeding conduit is in fluid communication with the central conduit of the flexible thermo-insulated hose.

The upper edge of the neck of the Dewar flask is sealed with a plug, which provided with a safety valve and a manometer, and serves at the same time for installation of the distal section of the central feeding conduit and the inlet section of the embedded heat exchanger (in the case of application of the bottle with pressurized gaseous medium).

In the other case, when the Dewar flask itself serves as a source of the gaseous medium, feeding cables of the electrical heater pass via the plug.

The distal end of the flexible thermo-insulated hose is provided with an outlet connection, which, in turn, is joined with a coupling unit intended for coupling with an associated proximal coupling unit of the cryoprobe.

The associated proximal coupling unit of the cryoprobe is preferably joined with a central feeding lumen, which supplies the gaseous medium into the internal space of the cryotip. The external shaft of the cryoprobe is preferably provided with a vacuum thermo-insulation. The exhausted gaseous medium is removed from the internal space of the cryotip through an annular channel formed between the vacuum thermo-insulation and the central feeding lumen and cleared out via an outlet connection in the coupling unit of the flexible hose.

The outlet connection of the flexible thermo-insulated hose and the outlet connection of its coupling unit are provided with two temperature sensors measuring the temperature of the supplied and cleared out gaseous medium.

Signals from these temperature sensors are directed to the control unit, which processes all data obtained from the temperature sensors and the mass flow rate gauge, and in accordance with the data recorded in the control unit shows on its display the calculated temperature of the cryotip and the estimated ice ball diameter, as a function of the cryosurgical procedure period and the cryotip temperature. In addition, the control unit can summarize the cold introduced into treated tissue during cryosurgical procedure and estimate on this basis the ice ball diameter.

In the case, when the pressurized gaseous medium is used as well as a thawing gaseous medium, there is a by-pass line providing immediate fluid communication of the bottle with the pressurized gaseous medium and the outlet connection of the flexible thermo-insulated hose.

### BRIEF DESCRIPTION OF THE SEVERAL VIEWS OF THE DRAWINGS

FIG. 1a demonstrates a cryosurgical system comprising a combination of a bottle with pressurized gaseous medium, an axial cross-sectional view of a Dewar flask with an embedded heat exchanger and a flexible thermo-insulated hose, which is coupled with a cryoprobe.

FIG. 1b demonstrates the cryosurgical system comprising combination of the bottle with pressurized gaseous medium, the axial cross-sectional view of the Dewar flask with the embedded heat exchanger, the flexible thermo-insulated hose, which is coupled with the cryoprobe and a by-pass line intended for thawing of a cryotip.

FIG. 1c demonstrates an axial cross-section of the Dewar flask with the embedded heat exchanger.

FIG. 1d demonstrates an enlarged axial cross-section of the cryoprobe, which is coupled with a distal section of the flexible thermo-insulated hose.

FIG. 2a demonstrates a cryosurgical system comprising combination of a bottle with pressurized gaseous medium, an axial cross-sectional view of a Dewar flask with an external heat exchanger of recuperative type and a flexible thermo-insulated hose, which is coupled with a cryoprobe.

FIG. 2b demonstrates the external heat exchanger of FIG. 2a.

FIG. 3a demonstrates a cryosurgical system operating by evaporation of liquid cryogen in the Dewar flask under a certain pressure; the obtained vapors of the cryogen are supplied as the pressurized gaseous medium at cryogenic temperature into a flexible thermo-insulated hose, which is coupled with a cryoprobe.

FIG. 3b demonstrates the Dewar flask and its siphon according to FIG. 3a.

FIG. 4a demonstrates a cryosurgical system comprising combination of a first bottle with pressurized gaseous medium and a second bottles with highly pressurized gas; the first one serves as a source of the pressurized gaseous medium, which cools the cryotip to cryogenic temperature; and the second one serves as a source of highly pressurized gas being expanded via a orifice tube in a heat exchanger; this figure demonstrates in addition a flexible thermo-insulated hose, which is coupled with a cryoprobe.

FIG. 4b shows an axial cross-section of the heat exchanger of FIG. 4a.

FIG. 5a demonstrates a cryosurgical system comprising: a bottle with pressurized gaseous medium and a combination of two cooling sources for lowering the temperature of the pressurized gaseous medium to a cryogenic temperature; a flexible thermo-insulated hose, which coupled is with a cryoprobe.

FIG. 5b shows an axial cross-section of the heat exchanger of FIG. 5a.

FIG. 5c shows an axial cross-section of the heat exchanging chamber of FIG. 5a.

### DESCRIPTION OF PREFERRED EMBODIMENTS

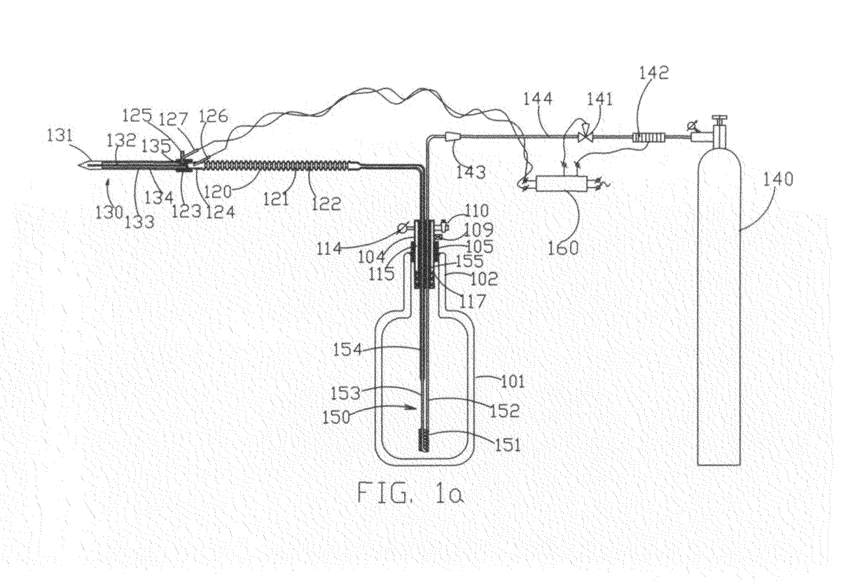

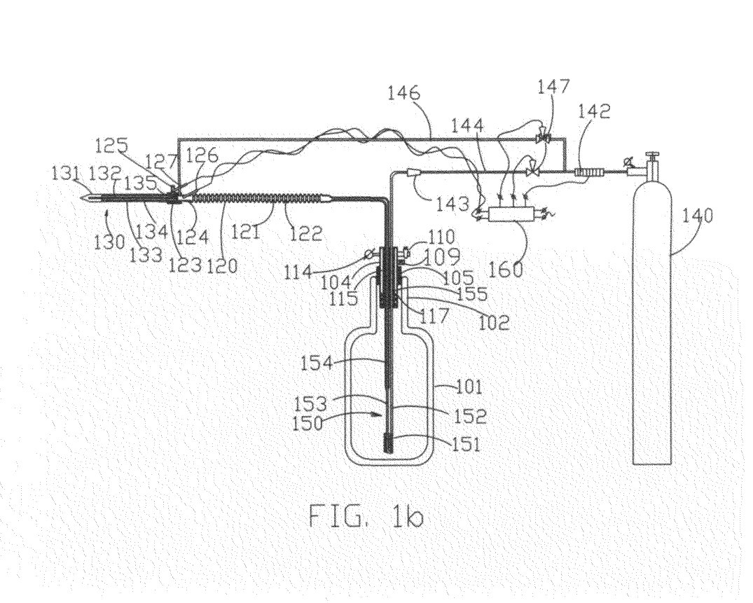

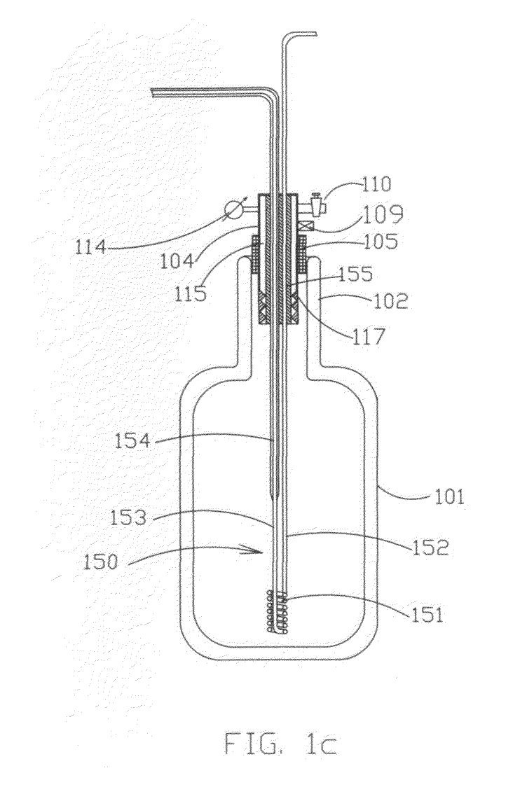

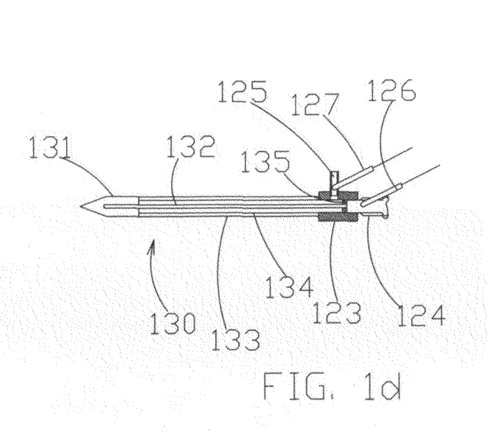

FIG. 1a shows an axial cross-sectional view of an exemplary Dewar flask 101 with a recuperative heat exchanger 150 installed in its neck according to preferred embodiments of the present invention, an axial cross-section view of a flexible hose 120 and an axial cross-sectional view of cryoprobe 130. The recuperative heat exchanger 150 and its auxiliary parts comprise a coil-type heat exchanger 151 itself with its inlet conduit 152 and an outlet conduit 153, which is provided with vacuum thermal insulation 154. The upper sections of the inlet conduit 152 and the vacuum thermal insulation 154 of the outlet conduit 153 are installed in bushing 155. Jacket 104 surrounds bushing 155 with gap 115 formed between them. The upper edge of jacket 104 is sealed with bushing 155 as shown.

There is also a seal for sealing jacket 104 to the Dewar flask 101, this seal is designed as is an annular rubber ring 105 installed on jacket 104 and inserted partially into neck 102 for holding bushing 155 in neck 102. Safety and relief valves 109 and 110 are installed on ports of the outer section of jacket 104. Jacket 104 also preferably features a pressure gauge 114, which is installed on its outer section for measuring internal pressure in the Dewar flask 101.

The lower section of the internal surface of jacket 104 is provided with an internal threading 117 with an internal diameter, which fits the outer diameter of bushing 155.

The distal external ends of the vacuum thermal insulation 154 of the outlet conduit 153 are joined with a vacuum thermal insulation 121 and a central lumen 122 of a flexible hose 120; in doing so the outlet conduit 153 and the flexible hose 120 have a common vacuum thermal insulation. The distal end of the central lumen 122 is provided with a coupling unit 123.

The coupling unit 123 is provided with an inlet connection 124 and an outlet connection 125.

Thermocouples 126 and 127 are installed in these inlet and outlet connections 124 and 125.

The coupling unit 123 is coupled with an associated coupling unit 135 of cryoprobe 130. Cryoprobe 130 comprises as well cryotip 131, central feeding lumen 132, an external shaft 133 and a vacuum thermal insulation 134.

Pressurized gaseous medium is provided into the inlet conduit 153 of the coil-type heat exchanger 151 from bottle 140 via a control valve 141, a mass flow rate gauge 142 and a coupling unit 143, which serves for coupling a supply line 144 with the inlet conduit 152.

A control unit 160 receives data from a mass flow rate gauge 142 and thermocouples 126, 127; it allows calculating temperature of cryotip 131 as it has been described in the summary of the invention.

FIG. 1b demonstrates the cryosurgical system with components, which correspond to similar components described with regard to FIG. 1a.

In addition, FIG. 1b comprises a by-pass line 146, which provides fluid communication via a control valve 147 between the supply line 144 and the inlet connection 124 of the coupling unit 123.

FIG. 1c demonstrates an axial cross-section of the Dewar flask with the embedded heat exchanger 150 and components, which correspond to similar components described with regard to FIG. 1a.

FIG. 1d demonstrates an enlarged axial cross-section of the cryoprobe 130 coupled with a distal section of the flexible thermo-insulated hose 120.

The distal section of the vacuum thermal insulation 121 and a central lumen 122 of the flexible hose 120 are provided with a coupling unit 123.

The coupling unit 123 is provided with an inlet connection 124 and an outlet connection 125.

Thermocouples 126 and 127 are installed in these inlet and outlet connections 124 and 125.

The coupling unit 123 is coupled with an associated coupling unit 135 of cryoprobe 130. Cryoprobe 130 comprises as well: cryotip 131; central feeding lumen 132; an external shaft 133 and a vacuum thermal insulation 134.

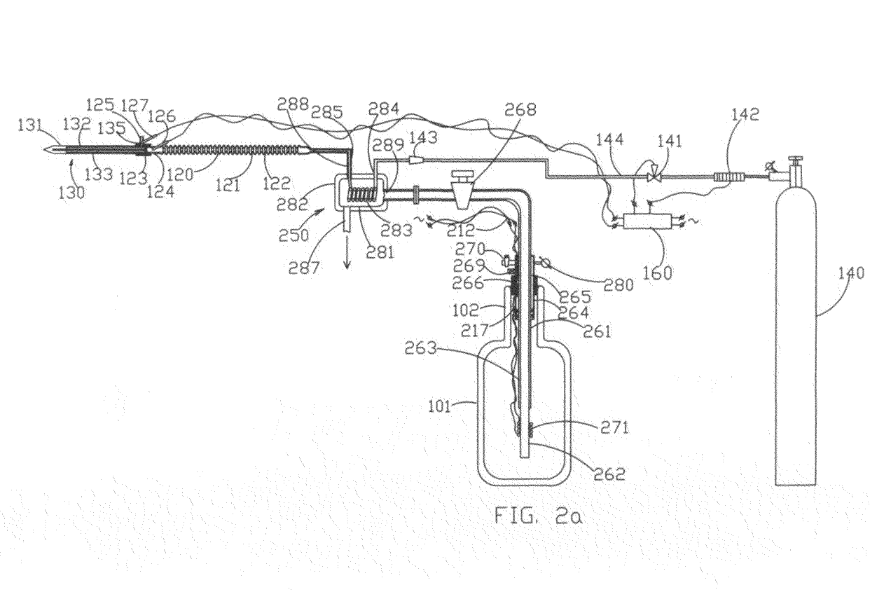

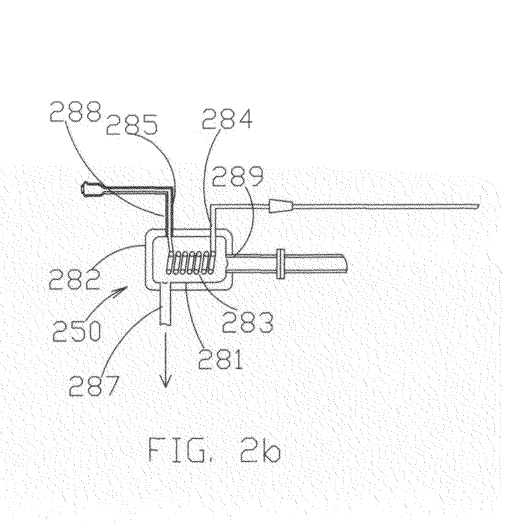

FIG. 2a shows: an axial cross-sectional view of an exemplary Dewar flask 101 with a recuperative heat exchanger 250 installed outside; an axial cross-section view of a flexible hose 120 and an axial cross-sectional view of cryoprobe 130.

The Dewar flask 101 comprises siphon 261 installed in its neck 102 according to preferred embodiments of the present invention, which is intended to be filled with a liquid cryogen. Siphon 261 comprises a feeding conduit 262 with a vacuum thermal insulation 263 of its middle and upper sections. There is jacket 264 surrounding the feeding conduit 262 and the vacuum thermal insulation 263 with gap 265 formed between them. The upper edge of jacket 264 is sealed with the vacuum thermal insulation 263 as shown. There is also a seal for sealing jacket 264 to the Dewar flask, and there is an annular rubber ring 266 installed on jacket 264 and inserted partially into neck 102 for holding siphon 261 in Dewar flask 101.

Also, preferably, a shut-off valve 268 is installed on the outer section of the feeding conduit 262. The shut-off valve 268 ensures supply control of the liquid cryogen.

In the preferred embodiment, safety and relief valves 269 and 270 are installed on ports of the outer section of jacket 264 for this purpose. Jacket 164 also preferably features a pressure gauge 280 for measuring internal pressure in the Dewar flask 101.

The lower section of the internal surface of jacket 264 is provided with an internal threading 217 with an internal diameter, which fits the outer diameter of the vacuum thermal insulation 263. An electrical heater 271 is placed on the lower section of the feeding conduit 262; this allows achieving operation pressure in the internal space of the Dewar flask 101.

An electrical cable 212 is supplying current to this electrical heater 271.

Gaseous-liquid mixture of cryogen is supplied by siphon 261 into a recuperative heat exchanger 250, which comprising a housing 281, a vacuum thermal insulation 282, a coil-type heat exchanging element 283 that is arranged in housing 281 and has inlet and outlet connections 284 and 285, and inlet and outlet connections 289 and 287, which serve for supplying the liquid-gaseous cryogen mixture from the dewar flask 101 and removal of evaporated cryogen from the internal space of the recuperative heat exchanger 250. The outlet connection 285 is provided with a thermal vacuum insulation 288, which is common with the vacuum thereto-insulation 121 of the flexible thermo-insulated hose 123.

A sub-system of gaseous medium supplying unit, a control unit 160, a flexible hose 120 and cryoprobe 130 are designed as the same units in FIG. 1a.

FIG. 2b demonstrates an axial enlarged cross-section the recuperative heat exchanger 250 of FIG. 2a.

It comprises: a housing 281; a vacuum thermal insulation 282; a coil-type heat exchanging element 283 that is arranged in housing 281 and has inlet and outlet connections 284 and 285; and inlet and outlet connections 289 and 287.

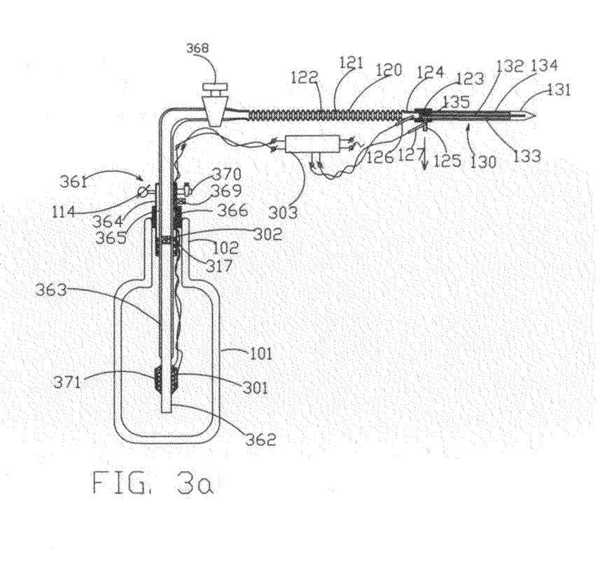

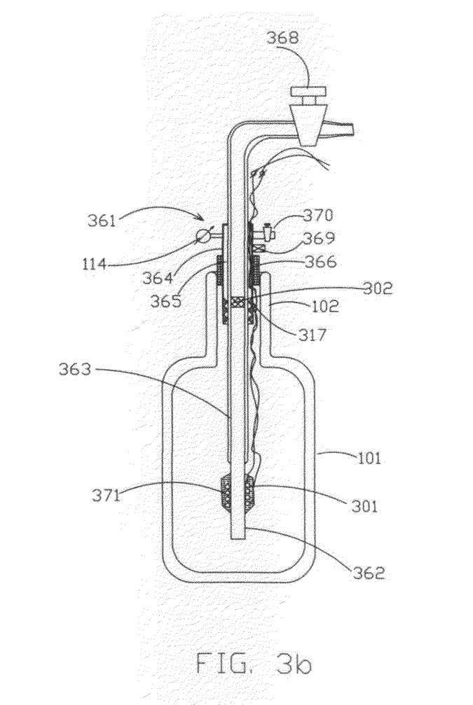

FIG. 3a demonstrates an axial cross-sectional view of an exemplary Dewar flask 101 with evaporation of liquid cryogen in this Dewar flask 101 under a certain pressure; in such a way the Dewar flask 101 with its siphon 361 provides the pressurized gaseous medium at cryogenic temperature. In addition, FIG. 3 shows: the axial cross-sectional view of the Dewar flask 101; an axial cross-section view of a flexible hose 120; an axial cross-sectional view of cryoprobe 130 and a control unit 303.

The Dewar flask 101 comprises siphon 361 installed in its neck 102 according to preferred embodiments of the present invention. Siphon 361 comprises a feeding conduit 362 with a vacuum thermal insulation 363 of its middle and upper sections. There is jacket 364 surrounding the feeding conduit 362 and the vacuum thermal insulation 363 with gap 365 formed between them. The upper edge of jacket 364 is sealed with the vacuum thermal insulation 363 as shown. There is also a seal for sealing jacket 364 to the Dewar flask, an there is an annular rubber ring 366 installed on jacket 364 and inserted partially into neck 102, for holding siphon 361 in Dewar flask 101.

Also, preferably a shut-off valve 368 is installed on the outer section of the feeding conduit 162. Safety and relief valves 369 and 370 are installed on ports of the outer section of jacket 364 for this purpose. Jacket 364 also preferably features a pressure gauge 114 for measuring internal pressure in the Dewar flask 101.

The lower section of the internal surface of jacket 364 is provided with an internal threading 317 with an internal diameter, which fits the outer diameter of the vacuum thermal insulation 363. An electrical heater 371 is placed on the lower section of the feeding conduit 362; this electrical heater is thermo-insulated on the outside by a thermal insulation 301. This allows achieving operation pressure in the internal space of the Dewar flask 101 and to evaporate cryogen at required rate.

The upper section of the feeding conduit 362 is provided with a demister 302, which separates droplets from gaseous-liquid mixture of cryogen with returning the droplets into the lower section of the feeding conduit 362.

The control unit 303 comprises a wattmeter measuring the heating power of the electrical heater 371 and allowing to calculate mass flow rate of gaseous medium without application of a mass flow rate gauge.

As in the case of FIG. 1a and FIG. 2a the control unit 303 receives data from thermocouples 126, 127; it allows calculating temperature of cryotip 131 as it has been described in the summary of the invention.

FIG. 3b demonstrates an axial cross-sectional view of the Dewar flask 101 with evaporation of liquid cryogen in this Dewar flask 101 under a certain pressure; in such a way the Dewar flask 101 with its siphon 361 provides the pressurized gaseous medium at cryogenic temperature. Siphon 361 comprises a feeding conduit 362 with a vacuum thermal insulation 363 of its middle and upper sections. There is jacket 364 surrounding the feeding conduit 362 and the vacuum thermal insulation 363 with gap 365 formed between them. The upper edge of jacket 364 is sealed with the vacuum thermal insulation 363 as shown. There is also a seal for sealing jacket 364 to the Dewar flask, and there is an annular rubber ring 366 installed on jacket 364 and inserted partially into neck 102 for holding siphon 361 in Dewar flask 101.

Also, preferably a shut-off valve 368 is installed on the outer section of the feeding conduit 162. Safety and relief valves 369 and 370 are installed on ports of the outer section of jacket 364 for this purpose. Jacket 364 also preferably features a pressure gauge 114 for measuring internal pressure in the Dewar flask 101.

The lower section of the internal surface of jacket 364 is provided with an internal threading 317 with an internal diameter, which fits the outer diameter of the vacuum thermal insulation 363. An electrical heater 371 is placed on the lower section of the feeding conduit 362; this electrical heater is thermo-insulated on the outside by a thermal insulation 301. This allows achieving operation pressure in the internal space of the Dewar flask 101 and to evaporate cryogen at required rate.

The upper section of the feeding conduit 362 is provided with a demister 302, which separates droplets from gaseous-liquid mixture of cryogen with returning the droplets into the lower section of the feeding conduit 362.

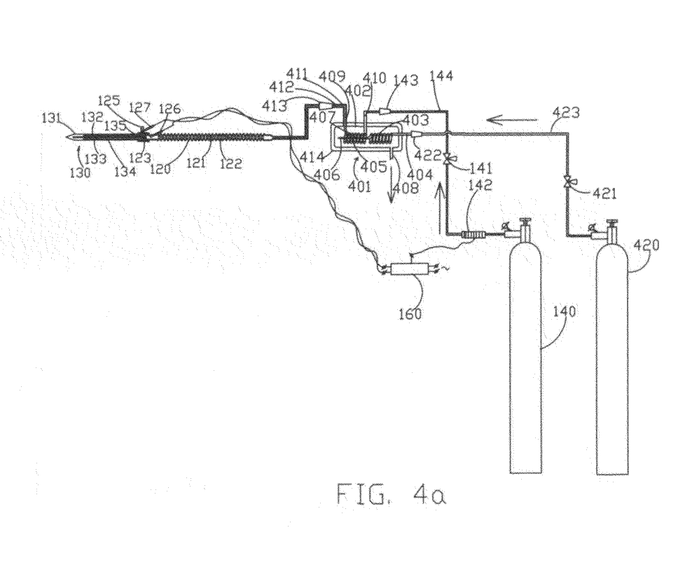

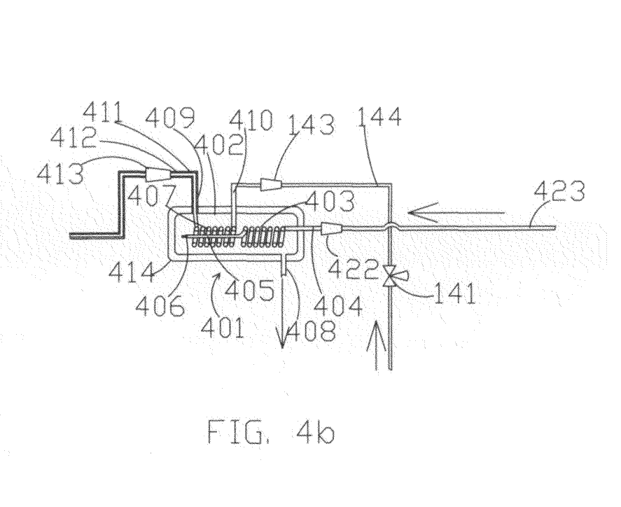

FIG. 4a demonstrates a cryosurgical system comprising a combination of a first bottle with pressurized gaseous medium and a second bottle with pressurized gas; the first one serves as a source of the pressurized gaseous medium, which cools the cryotip to cryogenic temperature; and the second one serves as a source of highly pressurized gas being expanded via an orifice tube in a heat exchanger serving for cooling the pressurized gaseous medium.

This heat exchanger 401 comprises an internal chamber 402 with a proximal coil-type heat exchanging unit 403, its inlet connection 404 and a middle lumen 405, which is terminated at its distal end with an orifice tube 406. The inlet connection 404 is in fluid communication with a second bottle 420 with pressurized gas via a coupling unit 422, line 423 and a control valve 421. An outlet connection 408 in the proximal section of the internal chamber 402 serves for clearing out the expended gas from its internal space.

In addition, there is a second distal coil-type heat exchanging unit 407, which is arranged in the internal chamber 402, with an inlet connection 410 and an outlet connection 409, this inlet connection is in fluid communication with the supply line 144. A coupling unit 143 serves for coupling the inlet connection 410 with the supply line 144 and the control valve 141.

The internal chamber 402 is provided with an outer vacuum thermal insulation 414.

In addition, the outlet connection 409 is provided with a vacuum thermal insulation 412.

A coupling unit 413 serves for coupling this outlet connection 409 with the flexible hose 120.

The flexible hose 120, cryoprobe 130 and a control unit 160 are designed in the same manner as these units in FIG. 1a.

FIG. 4b shows an enlarged axial cross-section of the heat exchanger 401 of FIG. 4a.

It comprises an internal chamber 402 with a proximal coil-type heat exchanging unit 403, its inlet connection 404 and a middle lumen 405, which is terminated at its distal end with an orifice tube 406. The inlet connection 404 is in fluid communication with the second bottle with pressurized gas via a coupling unit 422 and line 423. An outlet connection 408 in the proximal section of the internal chamber 402 serves for clearing out the expended gas from its internal space.

In addition, there is a second distal coil-type heat exchanging unit 407, which is arranged in the internal chamber 402, with an inlet connection 410 and an outlet connection 409; this inlet connection 410 is in fluid communication with the supply line 144. A coupling unit 143 serves for coupling the inlet connection 410 with the supply line 144 and the control valve 141. The internal chamber 402 is provided with an outer vacuum thermal insulation 414.

In addition, the outlet connection 409 is provided with a vacuum thermal insulation 412.

A coupling unit 413 serves for coupling this outlet connection 409 with the flexible hose.

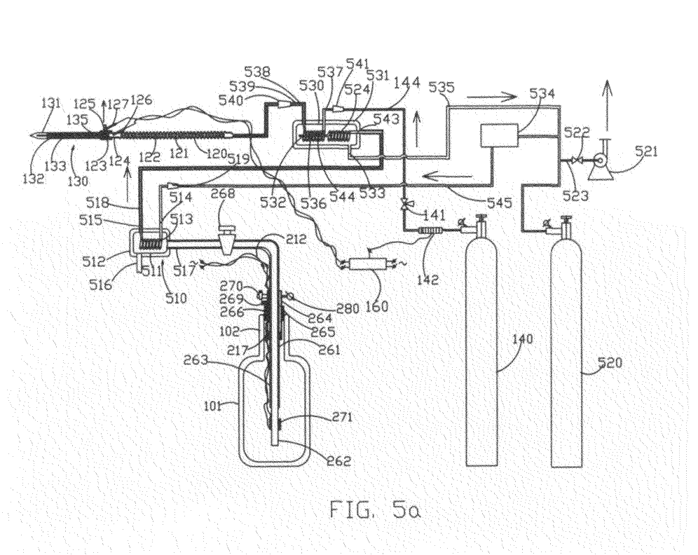

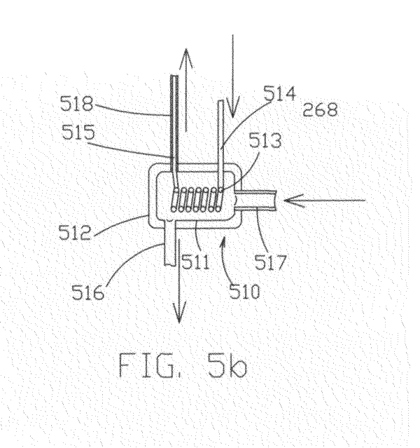

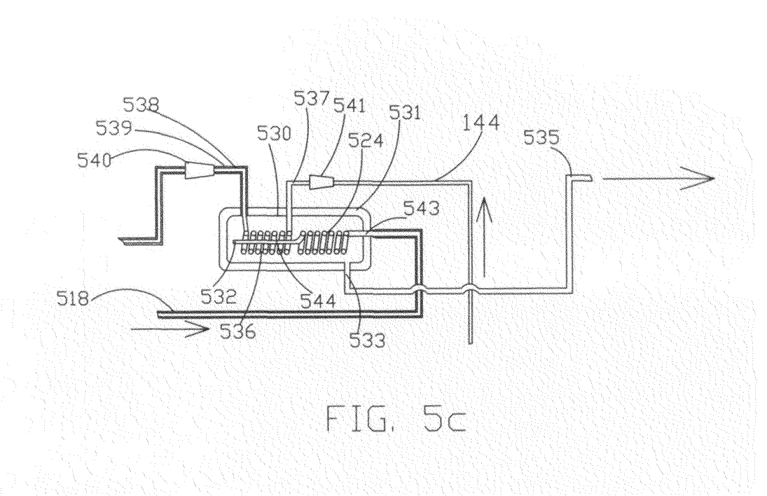

FIG. 5a demonstrates a cryosurgical system, which comprises: a bottle with pressurized gaseous medium and a combination of two cooling sources for lowering the temperature of the pressurized gaseous medium to a cryogenic temperature; an axial cross-section view of a flexible hose and an axial cross-sectional view of a cryoprobe.

The Dewar flask 101 serves for preliminary cooling the pressurized gas, which is circulating in a compression-expansion circuit; this Dewar flask 101 comprises siphon 261 installed in its neck 102 according to preferred embodiments of the present invention, which is intended to be filled with a liquid cryogen. Siphon 261 comprises a feeding conduit 262 with a vacuum thermal insulation 263 on its middle and upper sections. There is jacket 264 surrounding the feeding conduit 262 and the vacuum thermal insulation 263 with gap 265 formed between them. The upper edge of jacket 264 is sealed with the vacuum thermal insulation 263 as shown.

There is also a seal for sealing jacket 264 to the Dewar flask, an there is an annular rubber ring 266 installed on jacket 264 and inserted partially into neck 102, for holding siphon 261 in Dewar flask 101.

Also, preferably a shut-off valve 268 is installed on the outer section of the feeding conduit 262. The shut-off valve 268 ensures the control of the supply of the liquid cryogen.

In the preferred embodiment, preferably safety and relief valves 269 and 270 are installed on ports of the outer section of jacket 264 for this purpose. Jacket 264 also preferably features a pressure gauge 280 for measuring internal pressure in the Dewar flask 101.

The lower section of the internal surface of jacket 264 is provided with an internal threading 217 with an internal diameter, which fits the outer diameter of the vacuum thermal insulation 263. An electrical heater 271 is placed on the lower section of the feeding conduit 262; this allows achieving operation pressure in the internal space of the Dewar flask 101. An electrical cable 212 is supplying current to this electrical heater 271

Gaseous-liquid mixture of cryogen is supplied by siphon 261 into a recuperative heat exchanger 510, which comprising housing 511, a vacuum thermal insulation 512, a first coil-type heat exchanging unit 513, which is arranged in housing 511 and has inlet and outlet connections 514 and 515, and an outlet connection 516, which serves for removal of evaporated cryogen from the internal space of the recuperative heat exchanger 510. The outlet connection 515 is provided with a thermal vacuum insulation 518.

The compression-expansion circuit, which has been mentioned above comprises following units:

bottle 520, which serves for charging the compression-expansion circuit with a working gaseous medium, preferably, neon;

a vacuum pump 521 serving for preliminary purging the compression-expansion circuit from other gases;

a shut-off valve 522 which is installed in line 523 and provides fluid communication between the compression-expansion circuit and shut-off valve 522;

the first coil-type heat exchanging unit 513, which is arranged in the internal space of a heat exchanger 510 and serves for preliminary cooling the working gaseous medium (preferably—neon) to temperature below its inversion temperature; a coupling unit 519 serves for coupling the inlet connection 514 with a line providing fluid communication with compressor 534;

a second coil-type heat exchanging unit 524, which is arranged in a heat exchanging chamber 530 with a vacuum thermal insulation 531 and has an inlet connection 543 and a middle lumen 544, which is terminated at its distal end with an orifice tube 532; the expanded working gaseous medium is cleared out from the heat exchanging chamber 530 through an outlet connection 533, which is disposed in the proximal section of the heat exchanging chamber 530, and is directed into compressor 534 by line 535;

a third coil-type heat exchanging unit 536 is arranged in the distal section of the heat exchanging chamber 530 and serves for cooling the pressurized gaseous medium which is supplied into this third coil type heat exchanging unit 535 via an inlet connection 537 and is removed from the coil type heat exchanging unit 536 via an outlet connection 538, which is provided with a vacuum thermal insulation 539.

The pressurized gaseous medium is provided into the third coil type heat exchanging unit 536 from bottle 140 via a control valve 141, a mass flow rate gauge 142 and a coupling unit 541, which serves for coupling a supply line 144 with the inlet connection 537 of the third coil type heat exchanging unit 536.

A coupling unit 540 serves for coupling the outlet connection 538 with the flexible hose 120.

The flexible hose 120, the control unit 160 with its associated measuring means and cryoprobe 130 are designed in the same manner as these units in FIG. 1a.

FIG. 5b shows an axial cross-section of the heat exchanger 510 with the same units as in FIG. 5a.

It comprises: the first coil-type heat exchanging unit 513, which is arranged in the internal space of the heat exchanger 510 and serves for preliminary cooling the working gaseous medium (preferably—neon) to temperature below its inversion temperature; the inlet connection 514 is in fluid communication with the compressor.

FIG. 5c shows an axial cross-section of the heat exchanging chamber 530 with the same units as in FIG. 5a.

It comprises: the second coil-type heat exchanging unit 524, which is arranged in the heat exchanging chamber 530 with the vacuum thermal insulation 531 and has the inlet connection 543; the middle lumen 544, which is terminated at its distal end with the orifice tube 532; the expanded working gaseous medium is cleared out from the heat exchanging chamber 530 through the outlet connection 533, which is disposed in the proximal section of the heat exchanging chamber 530 and is in fluid communication with line 535.

1. A cryosurgical system consisting of:
a source of pressurized gaseous medium at cryogenic temperature; said source of pressurized gaseous medium is provided with means for measuring mass flow rate pressurized gaseous medium;
a cryoprobe comprising an external shaft, a central feeding lumen, a thermal insulation of said external shaft, a cryotip and a proximal coupling unit; the gap between said central feeding lumen and said thermal insulation forms an annular channel intended for return flow of said pressurized gaseous medium;
a flexible thermo-insulated hose with an internal conduit; a proximal end of said internal conduit is coupled with said source of pressurized gaseous medium and a distal end of said internal conduit is terminated with an associated coupling unit; said associated coupling unit comprises an internal duct for passage of said pressurized gaseous medium into said central feeding lumen and an outlet connection for clearing out said pressurized gaseous medium after its passage through said central feeding lumen, the internal space of said cryotip and said annular channel;
two temperature sensors, which are installed in said associated coupling unit of said flexible thermo-insulated hose; the first one is installed in said internal duct and the second one—in said outlet connection;
a control unit, which receives signals from said means for measuring mass flow rate of said pressurized gaseous medium and said temperature sensors and computing on the base of said signals an estimation of a real time ice ball diameter formed around said cryotip and operation temperature of said cryotip.

2. A cryosurgical system as claimed in claim 1, wherein the pressurized gaseous medium is pressurized gaseous helium.

3. A cryosurgical system as claimed in claim 1, wherein the source of pressurized gaseous medium at cryogenic temperature is designed as combination of a bottle with pressurized gaseous medium and a Dewar flask with an embedded heat exchanger; a line, which is fluid communicating said embedded heat exchanger and said bottle with pressurized gaseous medium, is provided with a flow mass rate gauge.

4. A cryosurgical system as claimed in claim 1, wherein there is a by-pass line providing immediate fluid communication of the bottle with the pressurized gaseous medium and the internal duct of the associated coupling unit

5. A cryosurgical system as claimed in claim 1, wherein the source of pressurized gaseous medium at cryogenic temperature is designed as combination of a bottle with pressurized gaseous medium and a Dewar flask with an external heat exchanger of recuperative type; said pressurized gaseous medium is supplied from the bottle into said external heat exchanger and at the same time, liquid-gaseous mixture of cryogen from said Dewar flask is supplied into said external heat exchanger.

6. A cryosurgical system as claimed in claim 5, wherein the external heat exchanger is provided with a vacuum thermal insulation.

7. A cryosurgical system as claimed in claim 1, wherein the source of pressurized gaseous medium at cryogenic temperature comprises: two bottles with pressurized gases; the first one serves as the source of the pressurized gaseous medium and the second one serves as a source of highly pressurized gas; said source of pressurized gaseous medium at cryogenic temperature comprises as well a heat exchanger, which consists of, in turn, a counter-flow coiled heat exchanging unit terminated with orifice tube and a coil-type heat exchanger; expansion of said gas causes its cooling on account of Joule-Thomson effect with following cooling the pressurized gaseous medium in said counter-flow heat exchanging unit arranged in said heat exchanger.

8. A cryosurgical system as claimed in claim 1, wherein the source of pressurized gaseous medium at cryogenic temperature comprises: two bottles with pressurized gases and a thermo-insulated vessel with a liquid cryogen; said first bottle serves as the source of the pressurized gaseous medium and said second bottle with pressurized gas in combination with said thermo-insulated vessel with said liquid cryogen are incorporated in a thermodynamic circuit, which consists of following units:
a vacuum pump, which serves for purging said thermodynamic circuit from gases; said vacuum pump is in fluid communication with said thermodynamic circuit via a first shut-off valve;
a second shut-off valve, which is installed on a line providing fluid communication of said second bottle with said thermodynamic circuit; said second bottle with said second shut-off valve serve for charging said thermodynamic circuit with a working gas having sufficiently low condensation temperature for pressure in vicinity of the atmospheric pressure;
a first heat exchanger with a coil-type conduit in its internal space; said first heat exchanger serves for preliminary cooling the gas from said second bottle below its inversion temperature by supplying into the internal space of said first heat exchanger said cryogen in liquid gaseous form;
a compressor, which serves for pressurizing said working gas upstream of said first heat exchanger;
a second heat exchanger, which consists of, in turn, a counter-flow coiled heat exchanging unit terminated with orifice tube and a coil-type heat exchanger; expansion of said working gas causes its cooling on account of Joule-Thomson effect with following cooling the pressurized gaseous medium in said counter-flow heat exchanging unit arranged in said second heat exchanger.

9. A cryosurgical system as claimed in claim 8, wherein the working gas is neon.

10. A cryosurgical system as claimed in claim 8, wherein the first heat exchange is provided with a vacuum thermo-insulation.

11. A cryosurgical system as claimed in claim 8, wherein the second heat exchanger is provided with a vacuum thermo-insulation.

12. A cryosurgical system as claimed in claim 1, wherein the source of the pressurized gaseous medium is designed as a Dewar flask with a siphon; said siphon has a central feeding conduit and the lower internal section of said central feeding conduit is provided with an electrical heater; a demister is installed in the upper internal section of said central feeding conduit; the middle and upper section of said central feeding conduit are provided with a vacuum thermo-insulation; a shut-off valve is installed on the distal end of said central feeding conduit;
the control unit is provided with a wattmeter, which is measuring the rate of heating the liquid cryogen in said siphon by said electrical heater.

13. A cryosurgical system as claimed in claim 12, wherein the lower section of the central feeding conduit is provided with a layer of thermal insulation.

14. A cryosurgical system as claimed in claim 1, wherein the control unit executes, in addition, calculated estimations of the thawing process.
